(19) United States
(12) Patent Application Publication
(10) Pub. No.: US 2004/0204635 A1
(43) Pub. Date: Oct. 14, 2004

(54) Devices and methods for the annotation of physiological data with associated observational data

(76) Inventors: Tom D. Scharf, Palm Harbor, FL (US); John E. Scharf, Oldsmar, FL (US); Scot L. Johnson, Tampa, FL (US)

Correspondence Address: JONES DAY, 555 WEST FIFTH STREET, SUITE 4600, LOS ANGELES, CA, 90013-1025, US

(21) Appl. No.: 10/411,870

(22) Filed: Apr. 10, 2003

(51) Int. Cl. A61B 5/00; A61B 5/02

(52) U.S. Cl. 600/323; 600/504; 600/502

(57) ABSTRACT

The present invention provides methods and devices for capturing data pertaining to caregiver observations that further define, describe, or qualify associated physiological data. The present invention facilitates the integration of numerous forms of observational data with associated physiological data, displays the recorded physiological data suitably annotated with the associated observational data through a variety of customizable graphical user interfaces, and uses general purpose portable computing devices such as PDAs, laptops, pocket PCs, or other microprocessor based devices for interfacing with a plurality of physiological sensors. In one embodiment, the present invention comprises a sensor to generate a plurality of physiological data related to a physiological condition of the subject, an event module to capture a plurality of observational data associated with a physiological condition of the subject, a plurality of receivers to receive the physiological data and the observational data, a clock in data communication with the receivers to time stamp the physiological data and observational data, a memory in data communication with the clock to store the time-stamped physiological data and time-stamped observational data, and a display in data communication with the memory to display the time-stamped physiological data in a time relation with the observational data.

### FIELD OF THE INVENTION

The invention relates generally to physiological condition monitoring systems, and more specifically, to systems and methods that capture and integrate physiological data with associated observational data and further display the physiological and observational data through a plurality of graphical user interfaces.

### BACKGROUND OF THE INVENTION

Physiological monitors of various types are used in the health and medical fields to monitor various physiological parameters of human patients. These physiological monitors allow health and medical professionals, as well as other users, to determine the current status of particular physiological parameters and monitor those parameters over a period of time. This information is helpful in health and fitness management and medical treatment.

Traditionally, physiological monitors include special-purpose computing devices designed to measure parameters related to specific physiological conditions of a subject. These special-purpose monitors are often unable to accommodate the monitoring of additional physiological conditions and, therefore, are restricted in their scope of use.

Conventional monitoring systems are used by caregivers, such as doctors, nurses, technicians, other health professionals and family members, to monitor parameters related to the physiological state of a patient. In the course of such monitoring, caregivers typically record observations of certain physiological parameters, such as blood oxygen saturation level of hemoglobin in arterial blood, the volume of blood pulsation supplying a tissue, the rate of blood pulsation corresponding to each heart beat, blood pressure, blood glucose levels, metabolic rate, breathing flow and volume, body temperature, pregnancy related factors, and other physiological data, by making notations on paper or recording observations into a tape recorder. These observations often pertain to, are in support of, build on, or question the parameters detected by the monitors and provide additional information that may be helpful in judging and formulating a diagnosis of the subject from time to time.

However, such conventional approaches to recording a caregiver's observations in relation to detected physiological data are disadvantageous for several reasons. For example, it is practically difficult to correlate such observations and notes with the actual recorded physiological data of a patient. Devices and methods used for capturing the caregiver's observations are not integrated with systems used for monitoring the physiological state of the patient. A lack of communication between systems for generating physiological data and associated observational data, such as caregiver opinions, thoughts, and clinical observations, either directly or through a common central device, limits how such data can be presented, manipulated, and used to better track, diagnose, and treat a patient.

In addition to the aforementioned disadvantages, it is currently not feasible to integrate a plurality of monitored physiological parameters, possibly gathered from different physiological state monitors, with caregiver observations taken in different forms, such as audio, video, image and/or textual data.

Therefore, there is need to capture data pertaining to caregiver observations that further define, describe, or qualify the associated physiological data in a way that facilitates the integration of all forms of observational data with the associated physiological data.

There is an additional need for displaying the recorded physiological data suitably annotated with the associated observational data through a variety of customizable graphical user interfaces. Additionally, there is need to utilize general purpose portable computing devices such as personal digital assistants (PDAs), laptops, pocket PCs, or other microprocessor based devices for interfacing with a plurality of physiological sensors and systems for capturing clinical observations to provide a common platform for suitably manipulating the data.

### SUMMARY OF THE INVENTION

In one embodiment, the present invention is a system for associating physiological data of a subject with observational data. The system comprises a sensor to generate a plurality of physiological data related to a physiological condition of the subject, an event module to capture a plurality of observational data associated with a physiological condition of the subject, a plurality of receivers to receive the physiological data and the observational data, a clock in data communication with the receivers to time stamp the physiological data and observational data, a memory in data communication with the clock to store the time-stamped physiological data and time-stamped observational data, and a display in data communication with the memory to display the time-stamped physiological data in a time relation with the observational data.

Optionally, the sensor may be a finger-clip pulse oximetry sensor and, further optionally, may comprise a light to frequency converter. The physiological data collected preferably pertains to a plurality of blood flow characteristics including at least one of a blood oxygen saturation of hemoglobin in arterial blood of the subject, a volume of blood pulsation supplying a tissue of the subject, and a rate of blood pulsation corresponding to a heart beat of the subject.

The display of the time-stamped physiological data is shown in a time relation with the observational data by displaying visual markers of the time-stamped observational data in association with graphical representations of the time-stamped physiological data. Alternatively, the display of the time-stamped physiological data is shown in a time relation with the observational data by displaying, in tabular form, a plurality of visual markers of the time-stamped observational data in association with a portion of the plurality of physiological data.

In another embodiment, the present invention is a method for associating physiological data of a patient with observational data. The method comprises the steps of generating a plurality of physiological data, capturing a plurality of observational data, associating a time with the plurality of physiological data, wherein the time is indicative of when the physiological data was generated, to produce time-stamped physiological data, associating a time with the plurality of observational data, wherein the time is indicative of when the observational data was captured, to produce time-stamped observational data, storing the plurality of time-stamped physiological data and time-stamped observational data, and displaying visual markers of the time-stamped observational data in association with the time-stamped physiological data.

Optionally, the visual markers of the time-stamped observational data are displayed in association with graphical representations of said time-stamped physiological data. Optionally, the visual markers of the time-stamped observational data are displayed in association with portions of the time-stamped physiological data in a tabular form.

Accordingly, it is one object of the present invention to provide a general-purpose hand-held portable computing device that can be adapted to monitor a plurality of physiological conditions of a subject.

It is another object of the present invention to enable the annotation of recorded physiological data with associated observational data that represent a caregiver's observations and comments associated with a subject's physiological condition. Such information is used to further support and/or qualify the physiological characteristics of the subject with clinical observation and analysis.

It is yet another object of the present invention to display physiological data along with the respective trend graphs in a variety of configurations. It is still another object of the present invention to display physiological data suitably annotated with event data.

The advantages of the invention are illustrated through a specific implementation that uses a typical finger-clip pulse oximetry sensor to detect the blood-flow characteristics of the subject, an audio memo system that captures voice event data, and a (PDA) device that receives, timestamps, stores, processes, annotates and displays the physiological and voice data through a plurality of user-friendly graphical user interfaces (GUIs).

### BRIEF DESCRIPTION OF THE DRAWINGS

These and other features and advantages of the present invention will be appreciated, as they become better understood by reference to the following Detailed Description when considered in connection with the accompanying drawings, wherein:

FIG. 1 is a functional block diagram of one embodiment of an exemplary system for annotating physiological data with associated observational data;

FIG. 2 is a functional block diagram of one embodiment of an audio memo system;

FIG. 3 is a flow diagram describing one embodiment of a process for the acquisition and processing of the physiological and audio observational data;

FIGS. 4a-4c depict a plurality of exemplary graphical user interfaces for the display of physiological data;

FIGS. 5a-5c depict a plurality of graphical user interfaces for the customization of data displays;

FIGS. 6a-6c depict one form of exemplary graphical user interfaces for the display of physiological data annotated with associated audio observational data;

FIG. 6d depicts another form of exemplary graphical user interfaces for the display of physiological data annotated with associated audio observational data; and

FIG. 6e depicts another form of exemplary graphical user interfaces for the display of physiological data annotated with associated audio observational data.

### DETAILED DESCRIPTION OF THE INVENTION

The present invention provides methods and devices for capturing data pertaining to caregiver observations that further define, describe, or qualify associated physiological data. The present invention facilitates the integration of numerous forms of observational data with associated physiological data, displays the recorded physiological data suitably annotated with the associated observational data through a variety of customizable graphical user interfaces, and uses general purpose portable computing devices such as PDAs, laptops, pocket PCs, or other microprocessor based devices for interfacing with a plurality of physiological sensors. The present invention will be described with reference to aforementioned drawings. One of ordinary skill in the art would appreciate that the applications described herein are examples of how the broader concept can be applied.

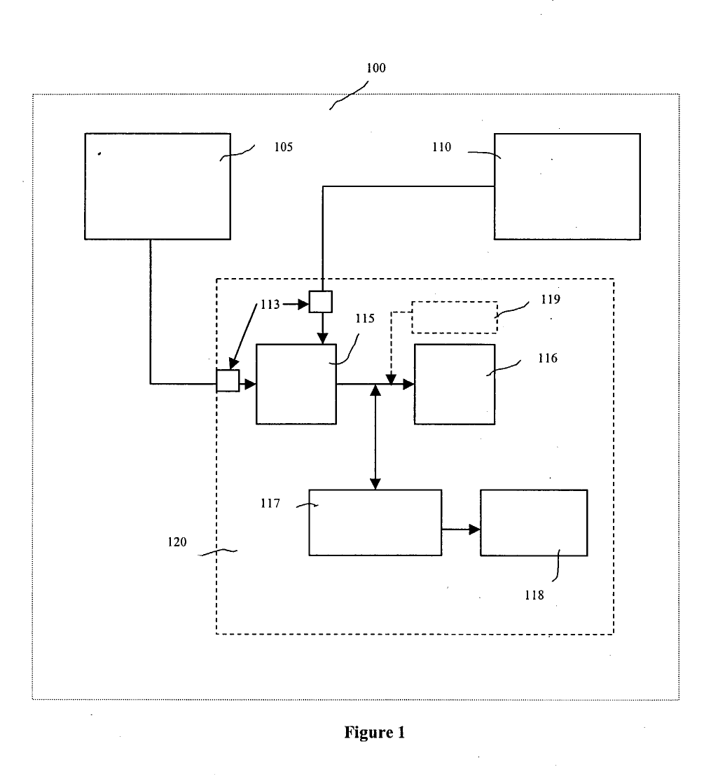

FIG. 1 shows a functional block diagram of an exemplary system 100 for annotating physiological data with associated observational data, including but not limited to audio data, video data, graphical images, and textual data. System 100 comprises at least one physiological sensing module 105 to measure a physiological characteristic of a subject, at least one event generation module 110 to capture audio, video, graphical, or textual observational information associated with the physiological data of the subject and a data processing module 120 to record, process, annotate and display data received from the modules 105 and 110 respectively.

The data processing module 120 further comprises a plurality of receivers 113 to receive physiological and observational data, clock module 115 to date and time stamp, on a real-time basis, the received physiological and observational data, memory module 116 to store the plurality of date and time stamped data, analysis module 117 to process data, and display module 118 to present annotated physiological data to a user by implementing a plurality of graphical user interfaces (GUIs). The data processing module 120 may also include an encryption module 119 to encrypt the physiological and observational data that are stored in memory 116.

The physiological sensing module 105 comprises a sensor to monitor the physiological condition of a subject. The sensor may be one of various types used in health and medical fields such as EKG monitors, exercise monitors such as pedometers, heart rate monitors, body temperature monitors, spirometers, electronic heart sound monitors, blood oxygenation and perfusion monitors, blood glucose monitors or any other suitable invasive or non-invasive physiological characteristic sensing system known to persons of ordinary skill in the art.

The event module 110 comprises systems used to capture audio, visual or textual observational information that represent a user's observations and comments associated with a subject's physiological condition. Such information is used to further support and/or qualify the physiological characteristics of the subject, being detected by the sensor module 105, with clinical observation and analysis. Physiological and event data from modules 105 and 110 are tagged by the clock module 115, with the date and time of data receipt, before being stored in the memory module 116 or further analyzed by the analysis module 117.

The encryption module 119 comprises means to receive the physiological and/or observational data and encrypt the data for storage. Encryption of the data may be achieved by algorithms known in the art that may, for example, allow access to the encrypted data by inputting a password. Alternatively, access to encrypted data may be allowed by a key. The encryption software may be stored in the memory module 116 or in other suitable location that is accessible by encryption module 119. By encrypting the collected data, the subject's physiological and/or observational data may be protected from unauthorized access or tampering.

Clock module 115, memory module 116, and analysis module 117 and, if applicable, the encryption module 119, can be implemented in various manners. For example, these elements can be implemented within a single integrated circuit, such as a DMC68HC 16 micro-controller from Motorola; within an application specific integrated circuit (ASIC); a digital signal processor or as any other suitable circuitry known to persons of ordinary skill in the art. The memory module 116 may comprise a random access memory (RAM), a FLASH memory, a programmable read only memory (PROM), an erasable PROM (EPROM), an electrically erasable PROM (EEPROM) or any other suitable electronic storage technologies known in the art.

One of ordinary skill in the art would appreciate the specific implementation of the modules depends upon the placement of the said modules within the overall structural implementation scheme of system 100. For example, in one preferred arrangement the modules 115, 116, 117 and 119 are implemented as an integrated data processing system. The data processing system comprises of a portable electronic microprocessor based device such as a laptop computer, personal digital assistant (PDA), electronic book, handheld computer or any other suitable handheld portable computing device known to persons of ordinary skill in the art. Thus, according to a preferred embodiment, the clock and memory modules 115 and 116 are standard hardware implementations available with a conventional PDA, with the analysis 117, encryption 119 and display 118 modules being implemented as a plurality of software applications and GUIs specific to computing and displaying the physiological condition being detected by the physiological sensing module 105. Similarly, while it is possible to incorporate the sensor module as an integrated unit built-into the data processing module, such as in a custom-built PDA, it is preferred that the sensor module be implemented as a detached stand-alone unit that can interface and communicate with any general purpose microprocessor based computing device through physical connections or wireless transmission, such as radio frequency transmissions.

Since such a computing device may communicate with a plurality of sensor modules attached to a plurality of patients, it is preferred that each sensor module have a unique identification (sensor ID) and be capable of reading, accepting, or otherwise receiving a unique identification tag of a patient/user (patient ID). While the sensor ID can be incorporated into the sensor module at the time of manufacture, the patient ID can be recorded into the computing device either manually by the user, by scanning a bar code tag associated with the patient or by any other suitable means known to persons of ordinary skill in the art. In a preferred embodiment, during communication, the sensor module transmits the unique ID and type of the sensor to the computing device, functioning as the data processing module 120 of FIG. 1, which is associated with the corresponding patient ID for data integrity.

According to one embodiment of the system 100, the module 105 is a finger-clip pulse oximetry sensor, the event module 110 is an audio memo system configured to capture a user's audio narrations and the data processing system 120 is a general purpose PDA implementing a pulse oximetry software application with related GUIs. A pulse oximetry sensor is used for generating signals related to blood flow characteristics including, but not limited to, the blood oxygen saturation of hemoglobin in arterial blood, and the rate of blood pulsation corresponding to each heart beat of a subject. A typical pulse oximetry sensor comprises LED (light emitting diode) emitters, one at a red wavelength and one at an infrared wavelength, and a photodiode detector. The sensor is typically attached to a body of tissue of the subject, such as an adult subject's finger or an infant subject's foot. Electromagnetic radiation from the emitters within the sensor are transmitted through the finger or reflected from finger tissue and subsequently detected by the photodiode detector.

For computing various blood flow characteristics of the subject, the photodiode-generated signals are conveyed to the data processing module 120 of FIG. 1 that, in the present embodiment, is a portable electronic microprocessor based device such as a PDA incorporating a pulse oximetry software application for further computation and display of data. One of ordinary skill in the art would appreciate the operation of a finger-clip pulse oximetry sensor, its signal processing capabilities, and the type of data transmitted to a monitoring station. For an exemplary system, reference is made to a preferred pulse oximetry sensor, referred to as Dolphin ONE™, designed and sold by Dolphin Medical, Inc. The preferred pulse oximetry sensor uses a light to frequency converter in the sensor to transform a detected signal to a digital signal at the point of detection.

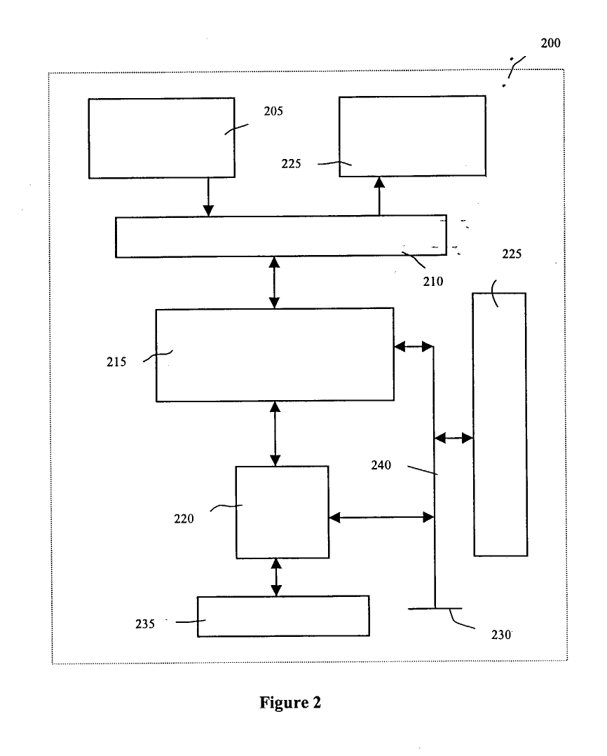

FIG. 2 shows a functional block diagram of an exemplary audio memo system 200, in accordance with one embodiment of the event generation module 110 of FIG. 1. In an exemplary arrangement, the audio memo system 200 is a stand-alone device capable of communicating data with an external computing device through a parallel data port 230 and where a microprocessor or micro-controller unit 220 controls operations of the system 200. The microprocessor unit 220 is connected to a digital signal processing (DSP) circuit 215 and exchanges command and data messages with the DSP 215. The DSP 215 is, in turn, connected to a circuit 210 that performs analog-to-digital (A/D) and digital-to-analog (D/A) signal conversion functions. System 200 also has a microphone 205 and speaker 225 that are connected to the A/D and D/A circuit 210. A multi-bit signal bus 240 interconnects the microprocessor unit 220 with the DSP 215, a memory interface 225 and a parallel data port 230 by which data may be exchanged with a computing device such as a PDA or a personal computer. The memory interface 225 comprises an embedded memory device and/or a removable memory card. The removable memory card may be used primarily for storing voice files and the embedded memory may be used primarily for program and working memory. However, these roles may be shared or reversed. One of ordinary skill in the art would appreciate that, in implementation, the data reception, transmission, and processing elements are accompanied by additional structural features that enable the control of recordation activities by a user.

The above-mentioned elements of the audio memo system 200 may be implemented using a number of components and devices as known to persons of ordinary skill in the art. For example, the microprocessor or micro-controller unit 220 may be implemented with the TMS370 family products of Texas Instruments Incorporated; the DSP circuit 215 may be implemented as a digital voice integrated circuit chip such as an ISD100AP; circuit 210 may be implemented with TCM320AC36 or TCM320AC37 voice-band audio processors manufactured by Texas Instruments Incorporated. The TCM320AC36 and TCM320AC37 voice-band audio processor (VBAP) integrated circuits perform the encoding (A/D conversion) and decoding (D/A conversion) together with suitable conditioning such as amplification, filtering, and antialiasing. The DSP circuit 215 implements compression and decompression of digitized voice data using algorithms known in the art.

While the elements of the system 200 have been shown implemented as discrete components and circuitry, it should be evident to persons of ordinary skill in the art that the same components may also be incorporated in a single chip implementation. Also, the memory interface 225 may comprise of random access memory (RAM), FLASH memory, programmable read only memory (PROM), erasable PROM (EPROM), electrically erasable PROM (EEPROM) for its embedded memory with the options for the removable memory unit expandable to include magnetic disk or tape, optical memory or any other suitable memory media known in the art. The use of a removable memory unit enhances the performance of the audio memo system 200. The storage capacity and, thus, the recording capacity of the system 200 may be greatly increased by the additional memory and the possibility of using multiple removable memory units.

In an alternate embodiment, the event generation module 110 of FIG. 1 may include a voice recognition command feature. The voice recognition command feature may be used to, for example, activate and deactivate the system 100 or give commands for entering observational information. The voice recognition command feature advantageously facilitates authorizing the operation of the system 100 to particular individuals according to his/her voice. The voice recognition command feature also advantageously allows the caregiver hands-free operation of the system 100 while caring for or observing the subject. The voice recognition command feature may be implemented via software or algorithm in the event generation module 110 as known to those skilled in the art.

In alternate embodiments the event generation module 110 of FIG. 1 may comprise digital video recorder such as a conventional digital camcorder to capture visual events/information related to a subject; digital radiographic scan images of a subject generated from X-ray radiography systems; textual diagnostic reports of the subject in the form of digital files or any other suitable system capable of providing contextual event/information, related to the physiological condition of a subject, as known to persons of ordinary skill in the art.

While the event module 110 may be external to module 120, in other embodiments, the module 110 may be built into and be an integral part of the data processing module 120. In a preferred embodiment the data processing module 120, that may be a handheld PDA, incorporates the audio memo system 200 of FIG. 2 with built in microphone, speaker and converter circuitry that is managed by a suitable voice acquisition and management software such as Microsoft's Media Player, Real Network's Real Player, Apple's Quicktime Player or any other similar software application known in the art. Similarly, the voice recognition command feature may also be integrated in the data processing module 120.

The functions of the micro-controller and DSP may be implemented in the form of audio processing software that resides on the PDA and utilizes the processing power of the PDA. The audio memo system 200 may further include an input interface 235 comprising actuating means to allow a user to control various functions of the system such as play, record, pause, stop, rewind, and forward. The actuating means may be implemented as physical push/press buttons, switches, dials or through a touch pad/screen interface such as that available in a typical PDA or any other suitable means known in the art.

Exemplary PDAs include the e740 Pocket PC, manufactured by Toshiba, Inc., and the IPAQ 3835 Pocket PC, manufactured by Compaq, Inc. The IPAQ comprises a Pocket PC 2002 operating system, 64 megabytes of built-in memory, a 240×320 color-reflective TFT LCD display, a USB cradle for connecting to a personal computer, an optional modem, handwriting recognition software, voice recognition command software, infrared data share capabilities, and voice recordation and functioning on a 206 MHz Intel 32 bit RISC processor with 32 MB flash ROM.

In a particular embodiment, operationally, the blood-flow characteristic and audio narration data from the sensor and audio memo systems respectively, are communicated to a PDA for further analysis, storage and display. A pulse oximetry software application, running on the PDA, computes a variety of blood flow related physiological parameters, annotates the resultant plurality of physiological parameters with the corresponding audio narration data, and generates a plurality of GUIs to display the processed and annotated data in a variety of configurations. In a preferred embodiment, the annotation of the physiological data with the corresponding audio data is implemented on the basis of the date and time stamps of the data and stored in encrypted form.

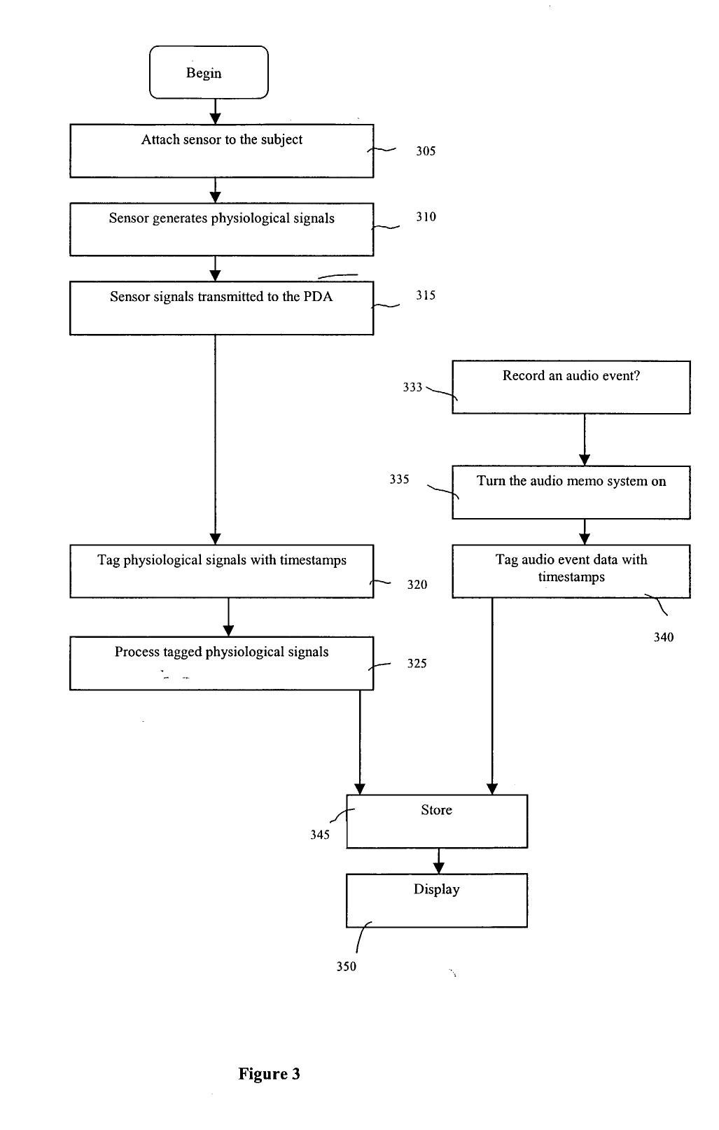

FIG. 3 shows a flow diagram describing an embodiment of one process for the acquisition and processing of the physiological and audio event data. An oximetry sensor is attached 305 to a subject. The sensor is in data communication with a PDA. At the command of the pulse oximetry software application, running on the PDA, the sensor begins generating 310 signals corresponding to certain blood flow characteristics of the subject. The signals are transmitted 315 to the PDA wirelessly or through connecting cables. The signals received at the PDA are then tagged 320 with the specific time when the data is recorded. Subsequently, the tagged physiological signals are processed 325 for computing various blood flow parameters to be displayed 330 through a plurality of graphical user interfaces.

Preferably, the processed and tagged signals of step 325 are stored 345 in memory for keeping a historical record of the physiological condition of the subject and retrieved for display 350 at a later time. In yet another embodiment, unprocessed tagged signals of step 320 may be initially stored in memory and then processed for display at a later time when required by the user.

In an alternate embodiment, the processed and tagged signals of step 325 and/or the unprocessed and tagged signal of step 320 is stored in step 245 in encrypted form to protect the data from unauthorized access.

Before, during, or after the sensor transmits 315 signals to the PDA, a caregiver determines 333 he or she wishes to document certain observations, diagnoses or any other information related to the physiological condition of the subject. In order to do so, the caregiver, or user, turns the audio memo system on 335, such as by pressing a button or switch that is preferably built into the PDA. The digitized voice data from the audio memo system is also tagged 340 with the date and time of generation of the voice data and subsequently stored 345 in the form of digital audio files for future reference and display/access 350. The tagged physiological and corresponding audio event data is obtained for a specific period of time continuously or intermittently as required by the user. Synchronization of sensor data with observational data is realized since the physiological data recorded by the sensing module is time-stamped by the same real-time clock that time-stamps the event data. The tagged data so obtained is finally displayed to the user through a plurality of GUI configurations.

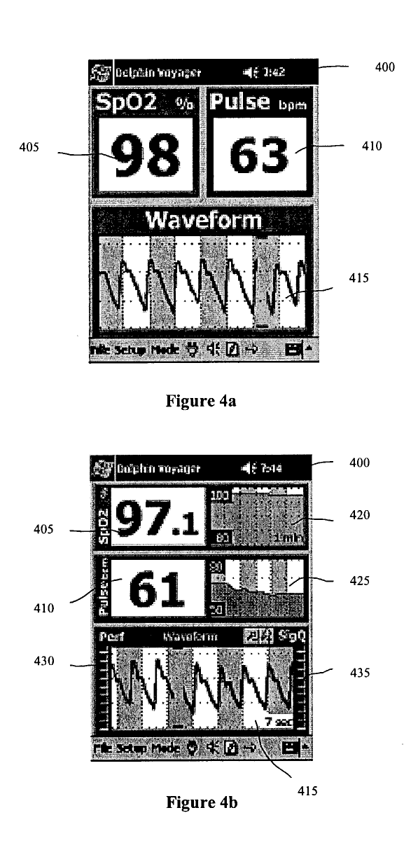

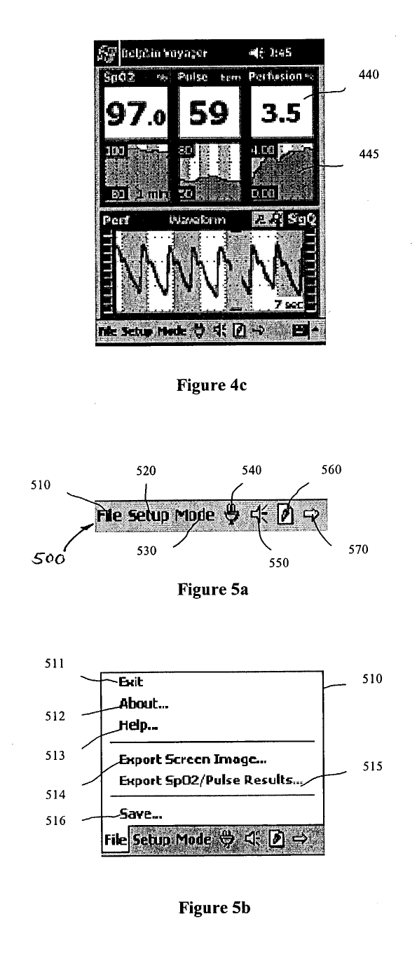

Referring to FIG. 4a, an exemplary GUI 400 is shown. The GUI 400 is partitioned into three sub-windows displaying the oxygen saturation (SpO2) level 405 and pulse rate 410 in alphanumeric notation while also showing a corresponding plethysmographic waveform 415 of the subject. The window 400 may be configured to display additional physiological parameters along with related trend graphs. For example, FIG. 4b depicts another exemplary GUI window 400 that not only shows alphanumeric values of SpO2 405 and pulse rate 410 but also portrays trend graphs 420 and 425 of the respective parameters. As shown, the trend-line graphs 420 and 425 depict the variation of the two parameters over a period of time. The parameters have been plotted on a time line based upon the date and time tags of the recorded physiological signals.

Additionally, contract and expand buttons/icons may be provided on the GUI that enable a user to change the time scale of the trend graphs. Alternatively a user may use the “tap-and-hold” feature in a PDA to change the time scale of the trend screens. The window 400 also provides the blood perfusion levels and the quality of detected pulse or physiological signals in the form of vertical bar graphs 430 and 435 respectively. FIG. 4c depicts yet another exemplary GUI view that displays a numeric perfusion index 440, as well as a trend screen 445 for perfusion in addition to the plurality of views displayed in FIG. 4b.

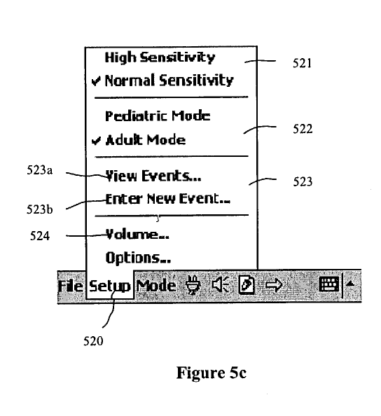

The plurality of GUI screens can be viewed, navigated and customized using control functions provided through a plurality of menus and buttons/icons shown in an exemplary toolbar 500 of FIG. 5a and that is incorporated in substantially all the views shown in FIGS. 4a through 4c. As shown in FIG. 5a, the toolbar 500 comprises file 510, setup 520 and mode 530 menus along with a group of short-cut buttons/icons comprising a battery level indicator 540 that displays the percentage of battery power remaining in the PDA/Pocket PC such that the indicator changes into a “plug” icon when an external power supply is connected to the pocket PC/PDA; an audio icon 550 that is used to enable or disable the speaker(s) built into the PDA and also set the levels of audio/alarm volumes; an event icon 560 that when clicked allows an audio, visual or textual event to be generated and inserted at the current physiological data readings; and a toggle icon 570 that is used to navigate through a plurality of views such as the screens shown in FIGS. 4a through 4c.

An exploded view of an exemplary file menu 510 is shown in FIG. 5b. The file menu 510 comprises an exit option 511 to quit the present oximetry software application so that a user may launch an alternative application appropriate for the physiological parameters to be analyzed or return to the normal functions of the PDA; about and help options, 512 and 513, to provide version and other functional information about the application; an option 514 that enables a user to save a picture of a screen, in the form of an image file, for archiving purposes; an export option 515 that saves desired SpO2, pulse rate and audio, visual and/or textual event data, along with the corresponding date and time tags, for future reference in various file formats, and a save option 516 that archives the sensor waveform data along with the readings and trend graphs of the physiological data annotated with any corresponding audio, visual and/or textual event data. The mode menu 530 also provides a group of options to manage and access data, such as a function to review stored physiological and event data files or functions to run or pause the acquisition and display of data.

An exploded view of an exemplary set-up menu 520 is provided in FIG. 5c. The set-up menu comprises a group of options 521 to adjust the sensitivity levels of the sensor for detecting the pulse signals, a group of options 522 to switch the working of the application between adult and pediatric modes; a group of options 523 for managing event data; an option 524 for adjusting volume for playback of audio event files and any other options to enhance the functionality and user-friendliness of the GUI that may seem appropriate to persons of ordinary skill in the art. The group of functions 523, for managing event data, may further comprise an option 523a for viewing event data and an option 523b for capturing the occurrence of a new event. The ‘enter new event’ option 523b can enable numerous functions, including enabling a user to start recordation of an observation and store it at a suitable destination. The ‘view events’ option 523a offers the user a plurality of GUI based screens to view audio, visual and/or textual observational data separately or as annotations integrated with the recorded physiological data.

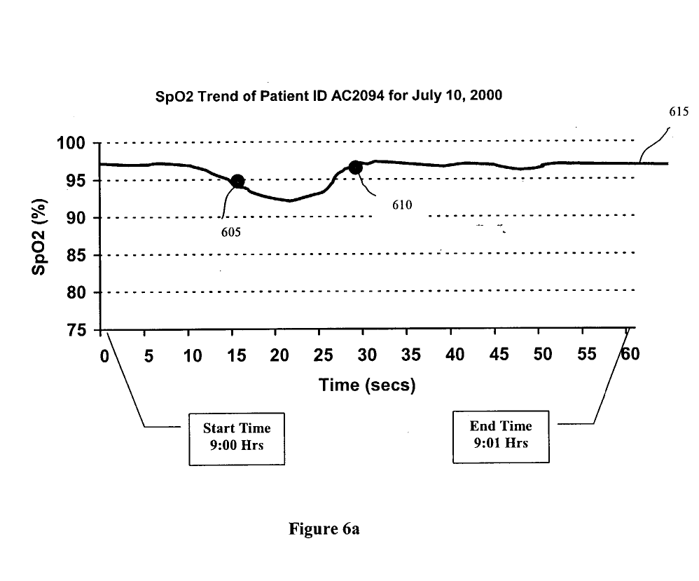

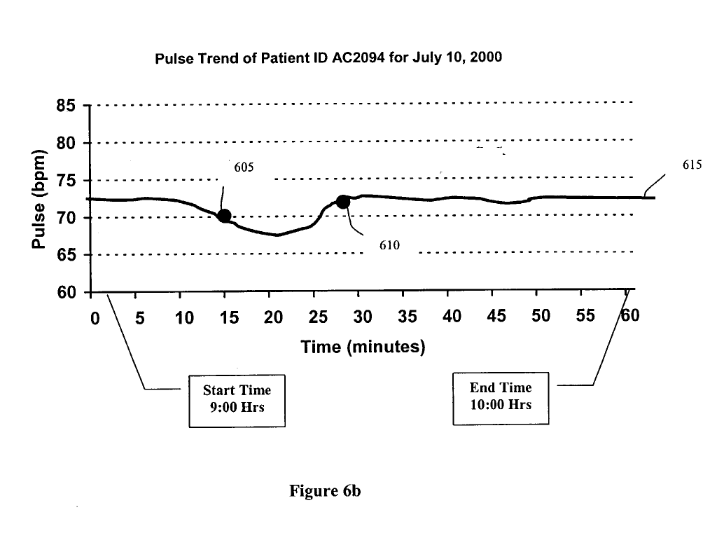

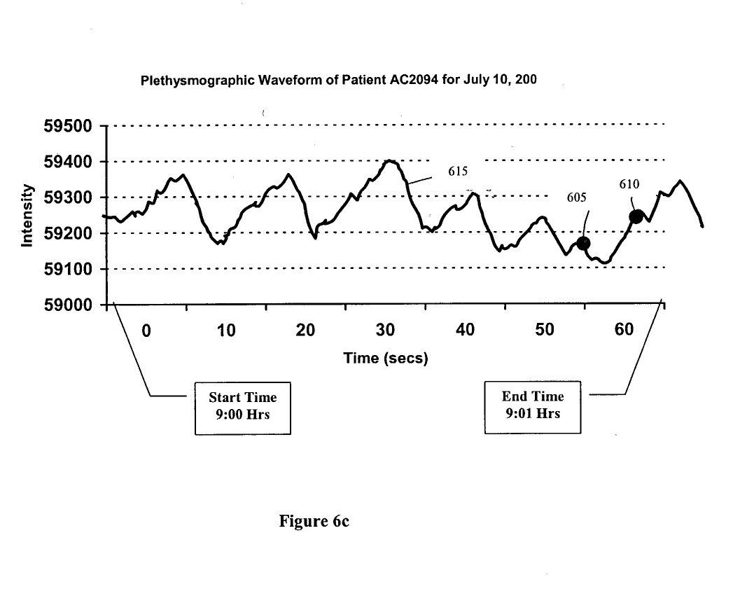

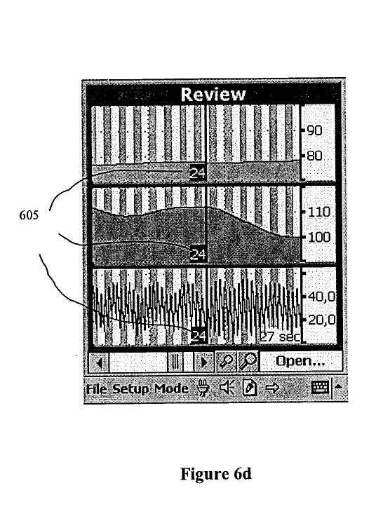

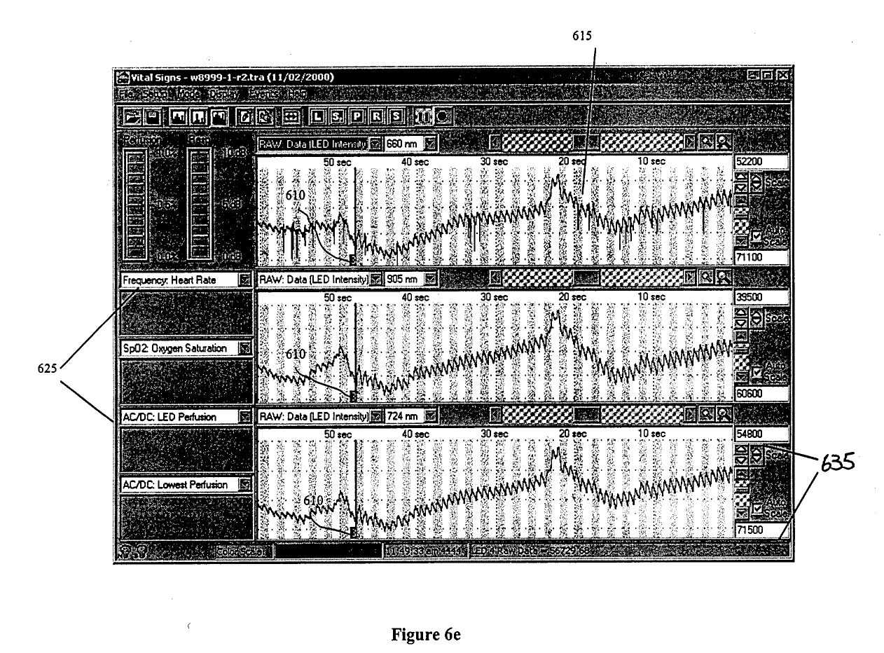

In accordance with a preferred embodiment, FIGS. 6a through 6e depict audio events superimposed on the physiological data trend graphs of SpO2, pulse rate and plethysmographic waveform respectively. As shown in FIG. 6a through 6c, the event markers or icons 605 and 610 indicate that the detected physiological data 615 of the subject have additional information or comments associated with them. The markers 605 and 610 are visual representations of event timestamps, thereby enabling a user to see when observational data was recorded relative to the physiological data. In the present embodiment, the event markers 605 and 610 are audio files that store voice comments, of a user, on a possible cause of sudden lowering and the subsequent restoration of the respective physiological condition of the subject. For example, the user may have clinically observed that the lowering of the various physiological parameters of the subject occurred because of the loosening of the sensor, and is therefore due to a motion artifact of the subject, rather than due to any real deterioration in the physiological condition of the subject. The user therefore recorded his observations in the form of a voice recordation that is shown marked as 605. Similarly the marker 610 may contain another voice file of the user that asserts the fact that the physiological parameters were found to be normal after adjusting the sensor.

While the markers 605 and 610 have been depicted as graphical circular dots, in FIGS. 6a through 6c, it should be evident to persons of ordinary skill in the art that the markers 605 and 610 can be depicted in a variety of ways such as numerals (1, 2, 3 and so on), characters (A, B, C, and so on), combination of numerals and characters or as graphic icons. Similarly, the markers 605 and 610 may be placed in a variety of different positions relative to the waveform and trend graphs. For example, the markers 605 and 610, in FIGS. 6d and 6e respectively, are shown as graphic numerals at the bottom of the respective graph adjacent to the time-line, instead of on the waveform or the trend line itself.

It is preferred that the graphical user interface, as shown in FIGS. 6d and 6e, provide a means by which different physiological data can be selected by a user. In one embodiment, a drop down menu 625 is provided that, when clicked on by a user, displays a list of physiological data that can be displayed. It is further preferred that the GUI concurrently provide access to multiple physiological data trends 615, along with relevant numerical data describing those trends 635.

It is preferred that the audio event files, depicted by the markers 605 and 610, be accessible to a user by simply tapping on the markers by a stylus, in the present embodiment of a PDA, or by clicking on the markers using a mouse in case of a portable computer. Thus in a preferred embodiment, the markers 605 and 610 are hyper-linked to the corresponding audio files stored in memory.

Although in the embodiments described above the audio event data has been shown superimposed on physiological data trend graphs and waveforms, one of ordinary skill in the art would appreciate that the data can be conveyed to and accessible by a user in a number of ways. In an alternative GUI interface the audio event files may be accessible through a click-down list that lists substantially all of the markers, representing the audio files recorded, together with a link to the time domain in the waveform and trend graphs that the audio file correlates to. The markers may be shown in a tabular format, as a series of observational events in a plurality of rows indicated by a listing number in one column, a time of recordation in a second column, and the status of certain measured physiological parameters, detected at the same time as that of the recordation, in a third column.

Preferably, users can observe the waveforms and trend graphs and access associated audio files and/or observe the list of audio files and access them, along with the time-corresponding physiological data. Observational or event data is not limited to audio files and may additionally comprise video, image, web and/or textual files.

While the system 100 of FIG. 1 has been described using a specific embodiment of pulse oximetry and audio memo systems, it should be appreciated by persons of ordinary skill in the art that any physiological condition monitoring sensor may be used in conjunction with any of the plurality of audio, video and/or imaging devices known in the art for generating the appropriate physiological and event data. Similarly, other add-ons to the system may be evident. For example, the system 100 may further be used to communicate with a centralized server or a personal desktop computer for long-term archiving or additional processing of the data. Thus, the digitized audio event data may be transcribed into text by transmitting the audio files to a personal computer running suitable automatic voice recognition algorithms known in the art. The transcribed data may then be accessible by means of magnetic media or by printed records generated by a printer.

Further, the system 100 may be configured with a lock-out feature that prevents access to some of the functions available to the system 100. For example, system 100 may be configured to allow only audio recording while preventing video recording. In another example, where the system 100 includes a PDA as the platform, non-monitoring functions such as personal program applications- and Internet access may be locked-out. The lock-out feature may be managed or controlled by, for example, an authorizing password. Such a lock-out feature may be achieved by using algorithms known to those skilled in the art. A lock-out feature advantageously provides a means to make use of a general purpose PDA while limiting the function for its intended use thereby discouraging theft or improper use of the device.

Other embodiments and modifications of the present invention will occur readily to those of ordinary skill in the art in view of these teachings. Such persons will appreciate and understand that the elements of the physiological data annotation system 100 may be arranged in other ways to produce similar results. For example, other types of computing devices can be used without departing from the scope of the invention. Therefore, this invention is to be limited only by the following claims, which include all such other embodiments and modifications when viewed in conjunction with the above specification and accompanying drawings.

What is claimed is:

1. A system for associating physiological data of a subject with observational data, comprising:
a sensor to generate a plurality of physiological data related to a physiological condition of the subject;
an event module to capture a plurality of observational data associated with a physiological condition of the subject;
a receiver to receive said physiological data and said observational data;
a clock in data communication with said receiver to time stamp the physiological data and observational data;
a memory in data communication with said clock to store the time-stamped physiological data and time-stamped observational data; and
a display in data communication with said memory to display the time-stamped physiological data in a time relation with said observational data.

2. The system of claim 1 wherein the sensor is a finger-clip sensor.

3. The system of claim 2 wherein the finger-clip sensor comprises a light to frequency converter.

4. The system of claim 1 wherein the physiological data pertains to a plurality of blood flow characteristics including at least one of a blood oxygen saturation of hemoglobin in arterial blood of the subject, a volume of blood pulsation supplying a tissue of the subject, and a rate of blood pulsation corresponding to a heart beat of the subject.

5. The system of claim 1 wherein the sensor is in data communication with a portable electronic microprocessor based device.

6. The system of claim 5 wherein the said at least one event generation module is integrated into the portable electronic microprocessor based device.

7. The system of claim 6 wherein the display and the clock module integrated into the portable electronic microprocessor based device.

8. The system of claim 1 wherein the plurality of observational data includes at least one of audio data, video data, graphical data, and textual data.

9. The system of claim 1 wherein the display of the time-stamped physiological data in a time relation with said observational data is achieved by displaying visual markers of the time-stamped observational data in association with graphical representations of the time-stamped physiological data.

10. The system of claim 1 wherein the display of the time-stamped physiological data in a time relation with said observational data is achieved by displaying, in tabular form, a plurality visual markers of the time-stamped observational data in association with a portion of the plurality of physiological data.

11. The system of claim 1 wherein the system further includes an encryption module in data communication with said memory to encrypt at least one of the time-stamped physiological data and the time-stamped observational data.

12. The system of claim 1 wherein the event module further includes a voice recognition command feature.

13. The system of claim 5 wherein the portable electronic microprocessor is configured to encrypt at least one of the time-stamped physiological data and the time-stamped observational data.

14. The system of claim 5 wherein the portable electronic microprocessor is further configured to include a function lock-out feature.

15. The system of claim 5 wherein the portable electronic microprocessor is further configured to include a voice recognition command feature.

16. A system for annotating physiological data of a subject with observational data, comprising:
at least one sensor to generate a plurality of physiological data related to a physiological condition of the subject; and
a portable electronic device in data communication with said sensor comprising at least one event module to capture a plurality of observational data associated with the physiological condition of the subject, a receiver to receive the physiological data, a clock to time stamp the plurality of physiological data and observational data, a memory module to store the plurality of time stamped physiological and time stamped observational data, a processor to process the physiological data and associate the physiological data in a time relation with the observational data, and a display for displaying the physiological and observational data.

17. The system of claim 16 wherein the at least one sensor is a finger-clip pulse oximetry sensor.

18. The system of claim 16 wherein the at least one event module is an audio memo system for recording audio generated by a caregiver.

19. The system of claim 16 wherein the portable electronic device is a personal digital assistant.

20. The system of claim 16 wherein the at least one event module is capable of capturing audio, video, image, or textual observational data.

21. The system of claim 16 wherein the processor is configured to encrypt the physiological and observational data.

22. The system of claim 16 wherein the processor is configured to include a function lock-out feature.

23. The system of claim 16 wherein the processor is configured to include a voice recognition command feature.

24. A method for associating physiological data of a patient with observational data, comprising the steps of:
generating a plurality of physiological data;
capturing a plurality of observational data;
associating a first time with the plurality of physiological data, wherein the first time is indicative of when the physiological data was generated, to produce time-stamped physiological data;
associating a second time with the plurality of observational data, wherein the second time is indicative of when the observational data was captured, to produce time-stamped observational data;
storing said plurality of time-stamped physiological data and said time-stamped observational data; and
displaying visual markers of said time-stamped observational data in association with said time-stamped physiological data.

25. The method of claim 24 wherein the visual markers of said time-stamped observational data are displayed in association with graphical representations of said time-stamped physiological data.

26. The method of claim 24 wherein the physiological data is generated by a finger-clip pulse oximetry sensor.

27. The method of claim 26 wherein the finger-clip pulse oximetry sensor comprises a light to frequency converter.

28. The method of claim 24 wherein the visual markers of said time-stamped observational data are displayed in association with portions of said time-stamped physiological data in a tabular form.

29. A method of annotating physiological data of a subject with observational data, the method comprising the steps of:
collecting physiological information from a subject by detecting light propagating through a body tissue of said subject;
processing said physiological information and computing a physiological value trend over a period of time;
collecting observational information related to an event; and
correlating said observational information related to said event with said physiological value trend based on time to supplement said physiological value trend with observational information.

30. The method of claim 29 wherein the step of correlating said observational information with said physiological value trend includes marking said physiological value trend with a marker to indicate that said physiological value trend includes said observational information related to said event.

31. The method of claim 29 wherein said observational information is collected in audio, video, graphical or textual form.

32. The method of claim 29 wherein said physiological value trend is at least one of an arterial oxygen saturation, pulse rate, or a volume of blood pulsation of said subject over said period of time.

33. The method of claim 29 further including the step of encrypting the physiological value trend and observational information to protect the physiological value trend and observational information from unauthorized access.

34. The method of claim 29 further including the step of displaying said observational information correlated with said physiological value trend.

35. A graphical user interface for associating physiological data of a subject with observational data, comprising:
a first area for displaying a first physiological parameter trend;
an event marker associated with said first physiological parameter trend for indicating that a supplemental information corresponding to said first physiological parameter trend is available; and
an access interface providing a user to select said event marker and display said supplemental information corresponding to said first physiological parameter trend.

36. The graphical user interface of claim 35 wherein said first physiological parameter trend is one of a blood oxygen saturation, a volume of blood pulsation supplying a tissue of a subject, and pulse rate.

37. The graphical user interface of claim 35 wherein said supplemental information is observational information.

38. The graphical user interface of claim 37 wherein the observational information is one of an audio data, video data, graphical data, and textual data.

39. The graphical user interface of claim 35 wherein said access interface provides one of a menu, button, and icon.

40. The graphical user interface of claim 35 wherein said access interface is accessible to the user by one of a stylus and mouse.

41. The graphical user interface of claim 35 wherein said first physiological parameter trend is determined by a processor configured to:
receive a light intensity signal detected from a light propagating through a body tissue of the subject; and
process said light intensity signal to compute said physiological parameter trend over a period of time.

42. A graphical user interface of claim 35 further including;
a second area for displaying a second physiological parameter trend;
an event marker associated with said second physiological parameter trend for indicating that a supplemental information corresponding to said second physiological parameter trend is available; and
an access interface providing a user to select said event marker and display said supplemental information corresponding to said second physiological parameter trend.
